(19) United States
(12) Patent Application Publication
(10) Pub. No.: US 2008/0285302 A1
(43) Pub. Date: Nov. 20, 2008

(54) DENTAL CURING LIGHT HAVING A SHORT WAVELENGTH LED AND A FLUORESCING LENS FOR CONVERTING SHORT WAVELENGTH LIGHT TO CURING WAVELENGTHS AND RELATED METHOD

(75) Inventors: Robert R. Scott, Riverton, UT (US); Dee Jessop, West Jordan, UT (US); Dan E. Fischer, Sandy, UT (US); Vasiliy Nosov, Moscow (RU)

Correspondence Address: WORKMAN NYDEGGER, 60 EAST SOUTH TEMPLE, 1000 EAGLE GATE TOWER, SALT LAKE CITY, UT, 84111, US

(73) Assignee: ULTRADENT PRODUCTS, INC., South Jordan, UT (US)

(21) Appl. No.: 12/145,115

(22) Filed: Jun. 24, 2008

Related U.S. Application Data
(63) Continuation of application No. 11/184,433, filed on Jul. 19, 2005.
Application No. 11/184,433 is a continuation-in-part of application No. 10/423,275, filed on Apr. 25, 2003.
Application No. 10/423,275 is a continuation-in-part of application No. 10/411,913, filed on Apr. 11, 2003, now Pat. No. 6,957,907.

(51) Int. Cl. A61B 1/06; A61C 3/00

(52) U.S. Cl. 362/573; 433/29

(57) ABSTRACT

A dental curing light includes a body, one or more LEDs configured to emit a first spectrum of light having a relatively short first peak wavelength (e.g., UV) disposed on the body, and a fluorescing lens that converts at least a portion of the first spectrum of light into a second spectrum of light having a second peak wavelength that is longer than the first peak wavelength. The second spectrum of light is selected so as to match the curing spectrum of a desired light-curable dental composition. In a preferred embodiment, the body comprises an elongate wand having a proximal end and distal end. The LEDs may be disposed at or near the distal end of the elongate wand.

### RELATED APPLICATIONS

The present application is a continuation of copending U.S. patent application Ser. No. 11/184,433, filed Jul. 19, 2005 and entitled “DENTAL CURING LIGHT HAVING A SHORT WAVELENGTH LED AND A FLUORESCING LENS FOR CONVERTING SHORT WAVELENGTH LIGHT TO CURING WAVELENGTHS AND RELATED METHOD”, and continuation-in-part of copending U.S. patent application Ser. No. 10/423,275, filed Apr. 25, 2003 and entitled “LIGHT EMITTING SYSTEMS AND KITS THAT INCLUDE A LIGHT EMITTING DEVICE AND ONE OR MORE REMOVABLE LENSES”, which is a continuation-in-part of U.S. patent application Ser. No. 10/411,913, filed Apr. 11, 2003 and entitled “ILLUMINATION APPARATUS HAVING A LIGHT-CONVERTING LENS FOR INCREASING VISUAL CONTRAST BETWEEN DIFFERENT ORAL TISSUES”, which issued as U.S. Pat. No. 6,957,907. The disclosure of each of the above applications and patent is incorporated by reference in its entirety.

### BACKGROUND OF THE INVENTION

1. The Field of the Invention

The present invention generally relates to the field of light curing devices and, more specifically, to light curing devices incorporating one or more light emitting diodes (LEDs). The light curing devices include a fluorescing lens to convert shorter wavelength light from at least one LED into one or more curing wavelengths.

2. The Relevant Technology

In the field of dentistry, dental cavities are often filled and/or sealed with photosensitive compounds that are cured by exposure to radiant energy, such as visible light. These compounds, commonly referred to as light-curable compounds, are placed within dental cavity preparations or onto dental surfaces where they are subsequently irradiated by light. The radiated light causes photosensitive components within the compounds to polymerize, thereby hardening the light-curable compounds within the dental cavity preparation or another desired location.

Existing light-curing devices are typically configured with a light source, such as a quartz-tungsten-halogen (QTH) bulb or an LED light source. QTH bulbs are particularly useful because they are configured to generate a broad spectrum of light that can be used to cure a broad range of products. In particular, a QTH bulb is typically configured to emit a continuous spectrum of light in a preferred range of about 350 nm to about 500 nm. Some QTH bulbs may even emit a broader spectrum of light, although filters are typically used to limit the range of emitted light to the preferred range mentioned above.

One reason it is useful for the QTH bulb to emit a broad spectrum of light is because many dental compounds cure at different wavelengths. For example, camphorquinone is a common photo-initiator that is most responsive to light having a wavelength of about 460 nm to about 470 nm. Other light-curable products, however, including many adhesives, are cured when they are irradiated by light wavelengths in the 350 nm to 400 nm range. Accordingly, QTH bulbs can be used to cure both camphorquinone initiated products as well as adhesives.

One problem with QTH bulbs, however, is that they generate a relatively high quantity of heat, making it impractical to place QTH bulbs on the portions of the light-curing devices that are inserted within the mouth of a patient. In particular, if the QTH bulbs were disposed at the tips of the light-curing devices, the heat generated by the QTH bulbs could burn or irritate the sensitive mouth tissues of the patient. Accordingly, the QTH bulbs are typically disposed remotely from the portion of the light-curing device that is inserted within a patient's mouth. The heat generated by QTH bulbs also represents wasted energy, which increases the power requirement to achieve a desired light intensity.

To channel and direct the light emitted by a QTH bulb to the desired location within a patient's mouth, existing curing lights must utilize light guides, such as fiber optic wands and tubular light guides, or special reflectors. Although fiber optic wands and reflectors are useful for their intended purposes, they are somewhat undesirable because they can add to the cost and weight of the equipment, thereby increasing the overall cost and difficulty of performing the light-curing dental procedures.

In an attempt to overcome the aforementioned problems, some light-generating devices have been manufactured using alternative light generating sources, such as light-emitting diodes (LEDs) which are generally configured to only radiate light at specific wavelengths, thereby eliminating the need for special filters and generally reducing the amount of input power required to generate a desired output of radiation.

LEDs are particularly suitable light sources because they generate much less heat than QTH bulbs, thereby enabling the LEDs to be placed at the tip of the curing lights and to be inserted directly within the patient's mouth. This is particularly useful for reducing or eliminating the need for light guides such as optical fiber wands.

One limitation of LEDs, however, is that they are only configured to emit a narrow spectrum of light. For example, a 460 nm LED or LED array will generally only emit light having a spectrum of 460 nm±30 mn. Accordingly, a light curing device utilizing a 460 nm LED light source will be well designed to cure camphorquinone initiated products, but will not be suitable for curing adhesives that are responsive to light in the 400 nm±30 nm range. Likewise, a light-curing device utilizing a 400 nm light source may be suitable to cure some adhesives, but will be unsuitable for curing camphorquinone initiated products.

In an attempt to overcome this limited utility, some light generating devices have been manufactured that include multiple LEDs configured to emit light at different wavelengths. However, because the different wavelengths of light are generated at different points (in contrast to a QTH bulb, for example), it can be difficult to produce sufficient (and substantially even) intensities of desired wavelengths across the full footprint of light emitted by the device. In other words, there are often “hot” and “cold” areas within the footprint of light generated with respect to any given wavelength and region of the footprint.

In view of the foregoing, it would be an advantage to provide a dental curing light including as little as one LED (although more than one LED could also be provided) that is still capable of providing a broad spectrum of output wavelengths for curing light activated dental compositions. It would be a further improvement if such a dental curing light were capable of providing more even intensities of any given wavelength across the full footprint of light emitted, as compared to typical dental curing lights including multiple LEDs (and thus multiple point sources of light) that each emit at a different peak wavelength.

### SUMMARY OF THE INVENTION

The present invention is directed to a dental curing light including a body, one or more LEDs configured to emit a first spectrum of light having a relatively short first peak wavelength (e.g., UV) disposed on the body, and a fluorescing lens that converts at least a portion of the first spectrum of light into a second spectrum of light having a second peak wavelength that is longer than the first peak wavelength. The second spectrum of light is selected so as to match the curing spectrum of a desired light-curable dental composition. In a preferred embodiment, the body comprises an elongate wand having a proximal end and distal end. The LEDs may be disposed at or near the distal end of the elongate wand.

The one or more LEDs configured to emit shorter wavelength light may emit any first peak wavelength desired that is shorter than the converted longer wavelength light exiting from the fluorescing lens. One such preferred embodiment includes at least one LED configured to emit shorter wavelength light having a wavelength between about 350 nm and about 410 nm. It is to be understood that the LED or LEDs may emit shorter wavelength light having a first peak wavelength that is shorter than 350 nm, if desired. For example, if the converted longer wavelength light has a second peak wavelength of about 380 nm (i.e., useful for curing various compounds including a proprietary initiator), then the one or more LEDs may be configured to emit light having a first peak wavelength shorter than about 380 nm.

In another embodiment, the one or more LEDs may emit first peak wavelengths that may be somewhat longer, but are still shorter than the converted longer wavelength light exiting from the fluorescing lens. One such preferred embodiment includes at least one LED configured to emit shorter wavelength light having a wavelength between about 350 nm and about 490 nm. It is to be understood that the LED or LEDs may emit shorter wavelength light having a first peak wavelength that is shorter than 350 nm, if desired. For example, if the converted longer wavelength light has a second peak wavelength of about 465 nm (i.e., useful for curing various compounds including camphorquinone as an initiator), then the one or more LEDs may be configured to emit light having a first peak wavelength shorter than about 465 mn.

The fluorescing lens converts at least a portion of the first spectrum of light into a second spectrum of light having a longer second peak wavelength. According to one embodiment, the longer wavelength light may have a second peak wavelength between about 350 nm and about 490 nm. In one such embodiment useful for curing various compounds including a proprietary UV activated initiator, the longer wavelength light may have a second peak wavelength between about 350 nm and about 410 nm. An alternative embodiment useful for curing various compounds including camphorquinone as an initiator may convert at least a portion of shorter wavelength light into longer wavelengths having a second peak wavelength between about 430 nm and about 490 nm, preferably between about 440 nm and about 480 nm.

The fluorescing lens may be selectively attachable and detachable from the body of the dental curing light. Such an embodiment may allow different fluorescing lenses to be interchanged as desired.

An inventive kit may include a dental curing light that includes a body and one or more LEDs disposed on the body. The one or more LEDs are configured to emit a first spectrum of light having a relatively short first peak wavelength. The kit also includes at least two different fluorescing lenses. A first fluorescing lens is capable of converting at least a portion of the first spectrum of light into a second spectrum of light having a second peak wavelength longer than the first peak wavelength. The second spectrum of light is selected so as to match the curing spectrum of a first light curable dental composition. A second fluorescing lens is capable of converting at least a portion of the first spectrum of light into a third spectrum of light having a third peak wavelength longer than the first peak wavelength. The third spectrum of light is selected so as to match the curing spectrum of a second light curable dental composition different from the first light curable dental composition.

For example, one of the fluorescing lenses may convert at least a portion of the shorter wavelength first spectrum of light into longer wavelengths having a peak wavelength between about 350 nm and about 410 nm. The other fluorescing lens may convert at least a portion of the shorter wavelength first spectrum of light into longer wavelength light, having a peak wavelength between about 430 nm and about 490 nm. The first and second fluorescing lenses may be selectively attachable and detachable from the body of the dental curing light so as to allow a user to interchange lenses as desired. The kit may further include one or more light curable dental compositions having curing characteristics that correspond to the curing spectrums produced by the fluorescing lenses.

A related inventive method involves the steps of: providing a dental curing light that includes a body and one or more shorter wavelength emitting LEDs disposed on the body; selecting a fluorescing lens that converts at least a portion of the emitted first spectrum of relatively short wavelength light into longer wavelength light having a spectrum selected so as to match the curing spectrum of a particular desired light-curable dental composition; attaching the selected fluorescing lens to the dental curing light; and using the dental curing light and attached fluorescing lens to cure the particular desired light-curable dental composition.

These and other benefits, advantages and features of the present invention will become more full apparent from the following description and appended claims, or may be learned by the practice of the invention as set forth hereinafter.

### BRIEF DESCRIPTION OF THE DRAWINGS

In order that the manner in which the above recited and other benefits, advantages and features of the invention are obtained, a more particular description of the invention briefly described above will be rendered by reference to specific embodiments thereof which are illustrated in the appended drawings. Understanding that these drawings depict only typical embodiments of the invention and are not therefore to be considered limiting of its scope, the invention will be described and explained with additional specificity and detail through the use of the accompanying drawings in which:

FIG. 1 illustrates a graph charting the spectral irradiance of a Quartz Tungsten Halogen (QTH) bulb, a 380 nm LED, a 430 nm LED, and a 455 nm LED;

FIG. 2 illustrates an exemplary dental curing light of the invention having an elongate wand body that includes one LED disposed at the distal end of the elongate wand of the dental curing light;

FIG. 3 illustrates a graph charting the spectral irradiance of a dental curing light of the invention including a 400 nm LED and a fluorescing lens that converts a portion of the shorter wavelength light to a longer wavelength having a peak of about 460 nm;

FIG. 4 illustrates another exemplary dental curing light that includes two shorter wavelength LED light sources disposed at the distal end of the elongate wand of the curing light; and

FIG. 5 illustrates an exemplary kit including a dental curing light and a plurality of different fluorescing lenses.

### DETAILED DESCRIPTION OF THE PREFERRED EMBODIMENTS

I. Introduction and Definitions

A detailed description of the invention will now be provided with specific reference to Figures illustrating various exemplary embodiments. It will be appreciated that like structures will be provided with like reference designations.

To help clarify the scope of the invention, certain terms will now be defined. The terms “LED” and “LED light source,” as used herein, generally refer to one or more LEDs, one or more LED arrays, or any combination of the above that is capable of generating radiant energy. The light emitted by an LED light source includes a limited spectrum of wavelengths with a peak wavelength that corresponds with the rating of the LED light source.

The term “footprint,” as used herein, refers to the cross-sectional shape of light emitted by a light emitting device. The dimensions of the footprint will typically vary according to the distance of the footprint from the light source and the angle at which the light is emitted by the light source. The general shape and dimensions of a footprint can generally be identified by placing a flat object in front of a light source and observing the area illuminated by the light source.

The term “spectrum of light” refers to light that is monochromatic or substantially monochromatic, as well as light that falls within a range of wavelengths. The term “wavelength”, when used in the context of the term “spectrum of light”, refers to either the actual wavelength of monochromatic light or the dominant or peak wavelength within a range of wavelengths.

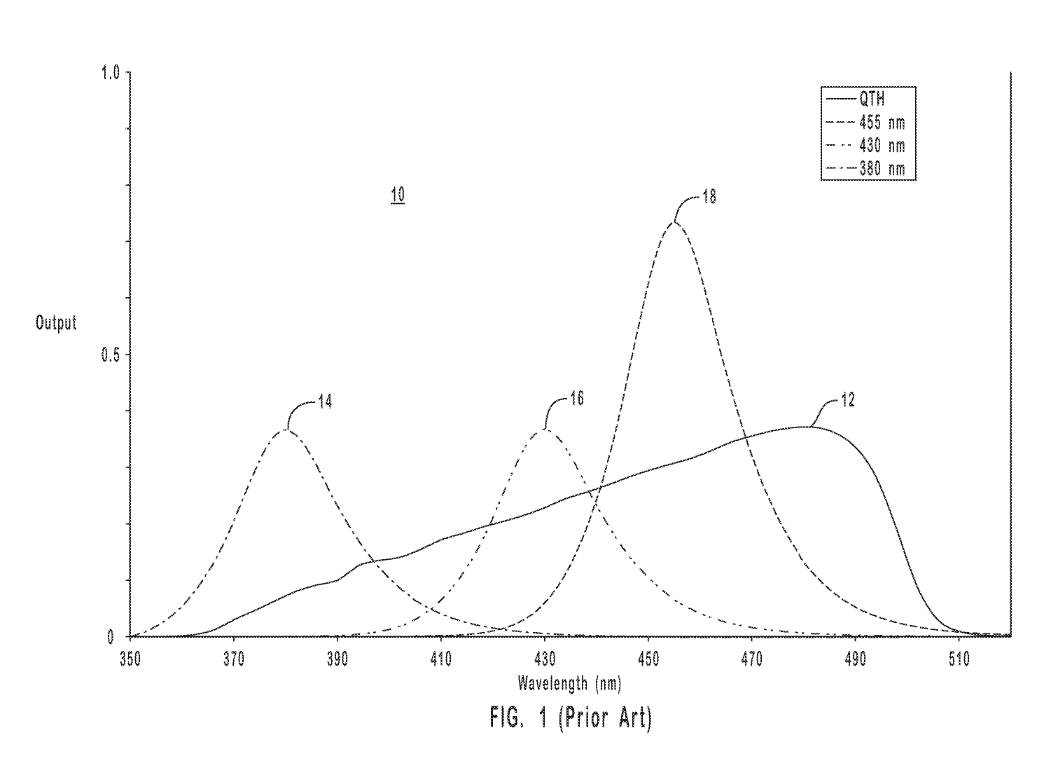

FIG. 1 illustrates a graph 10 that charts the spectral irradiance or light spectra emitted from by a quartz-tungsten-halogen (QTH) bulb, a 380 nm LED light source, a 430 nm LED light source, and a 455 nm LED light source. The values given in the y-axis are generic such that no specific representation as to the actual power output should be assumed.

As shown in FIG. 1, the QTH spectrum 12 ranges from about 360 mn to about 510 nm. The 380 nm LED spectrum 14 ranges from about 350 nm to about 430 nm, with the most intense output of light being within the range of about 360 nm to about 400 nm. The 430 nm LED spectrum 16 ranges from about 390 nm to about 480 nm, with the most intense output of light being within the range of about 410 nm to about 450 mn. The 455 nm LED spectrum ranges from about 410 nm to about 510 nm, with the most intense output of light being within the range of about 430 nm to about 490 nm.

Also shown, each of the individual LED spectra 14, 16 and 18 individually comprise only a portion of the spectral range of wavelengths emitted by QTH spectrum 12. Accordingly, the utility of the LED spectra 14, 16, and 18 is somewhat more specialized or limited than the spectral irradiance of the QTH spectrum 12. In particular, the QTH spectrum 12 can be used to cure adhesives that are responsive to light at about 350-410 nm (i.e., UV light), as well as camphorquinone initiated products that are responsive to light at about 430-490 nm (i.e., blue light). In contrast, none of the individual LED Spectra 14, 16 or 18 can be used to effectively cure both camphorquinone initiated products as well as adhesives that are activated by 350-410 nm light.

Accordingly, QTH bulbs have greater utility than individual LEDs from the standpoint of providing light in a broad spectrum. However, as mentioned above, the heat generated by QTH bulbs is undesirable and effectively prevents the QTH bulb from being placed on the portion of the light-curing device that is inserted within a patient's mouth, thereby requiring QTH bulb devices to be used with light-guides to direct the light to the desired location within a patient's mouth. In contrast, LED light sources can be placed at or near the ends of the light-curing devices and inserted within a patient's mouth. LEDs, however, emit only a narrow spectrum of light, effectively limiting their use to photo-curing a limited range of products, as compared to the broader range of products that can be cured using a QTH bulb.

The present invention is directed to a dental curing light including a body, one or more LEDs disposed on the body, the LED or LEDs being configured to emit a first spectrum of light having a relatively short first peak wavelength, and a fluorescing lens that converts at least a portion of the first spectrum of light into a second spectrum of light having a second peak wavelength that is longer than the first peak wavelength. The second spectrum of light is selected so as to match the curing spectrum of a desired light-curable dental composition. In a preferred embodiment, the body comprises an elongate wand having a proximal end and distal end. The LEDs may be disposed at or near the distal end of the elongate wand.

II. An Exemplary Dental Curing Light

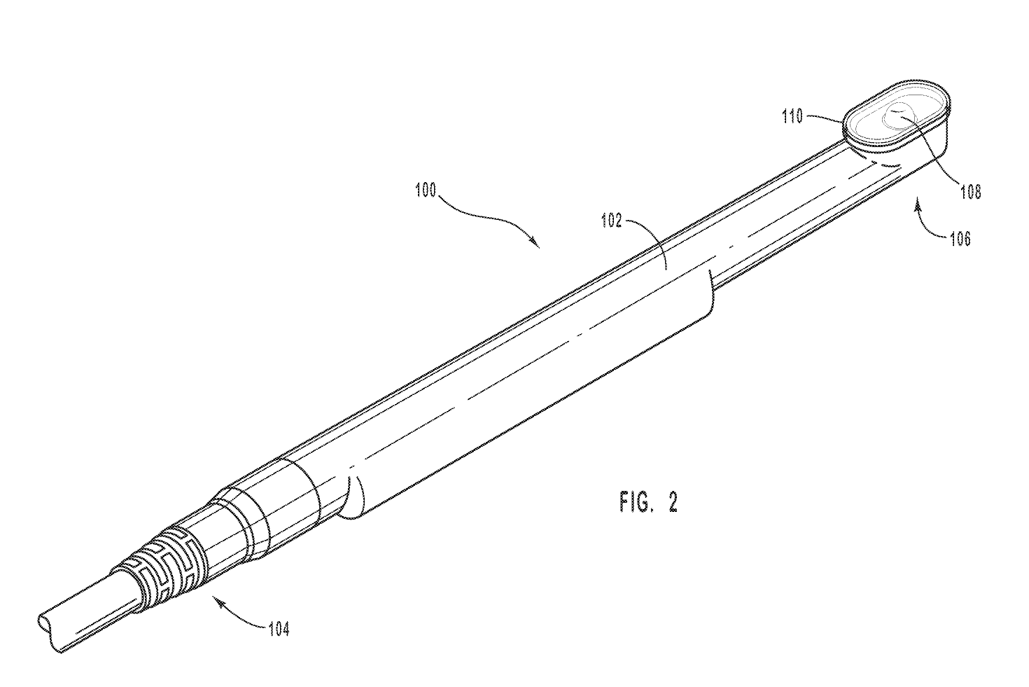

FIG. 2 illustrates an exemplary dental curing light 100. Dental curing light 100 includes a body 102 which may be configured as an elongate wand having a proximal end 104 and a distal end 106. Dental curing light 100 also includes at least one LED 108 disposed at or near the distal end 106 of elongate wand body 102. A fluorescing lens 110 that converts at least a portion of the first spectrum of light emitted from LED 108 into a second spectrum of light having a second peak wavelength that is longer than the first peak wavelength is located over LED 108. Fluorescing lens 110 may be selectively attachable and detachable from the dental curing light. The fluorescing lens is positioned over LED 108 so as to receive shorter wavelength light emitted by LED 108. Fluorescing lens 110 converts the shorter wavelength light of the first spectrum into longer wavelength light of a second spectrum. The second spectrum is selected so as to match the curing spectrum of a desired light-curable dental composition (e.g., camphorquinone initiated products or UV initiated adhesives).

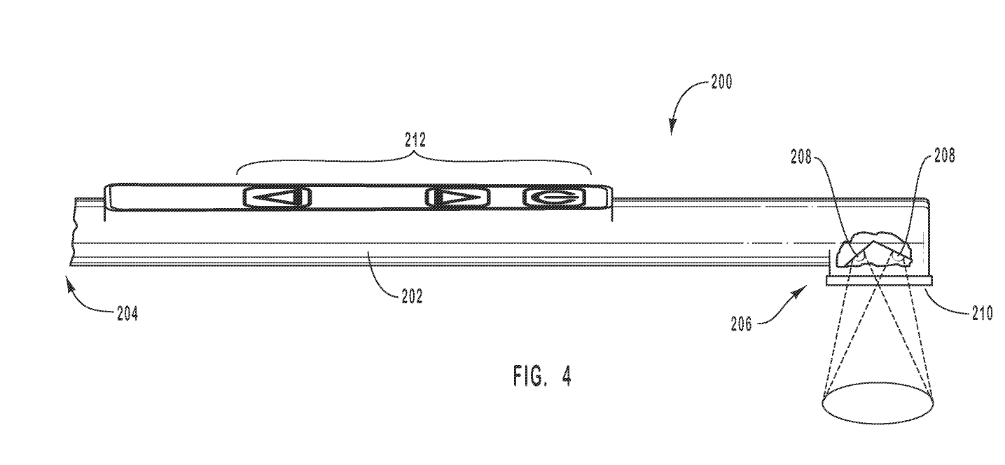

The dental curing light 100 may include controls disposed on elongate wand body 102 for selectively controlling operation of one or more LEDs 108. The controls may comprise any suitable control system. One illustrated embodiment, perhaps best seen in FIG. 4, includes multiple buttons (e.g., buttons 212) disposed on elongate wand body 102. Buttons 212 or another control system may allow activation of one or more LEDs 108 as desired.

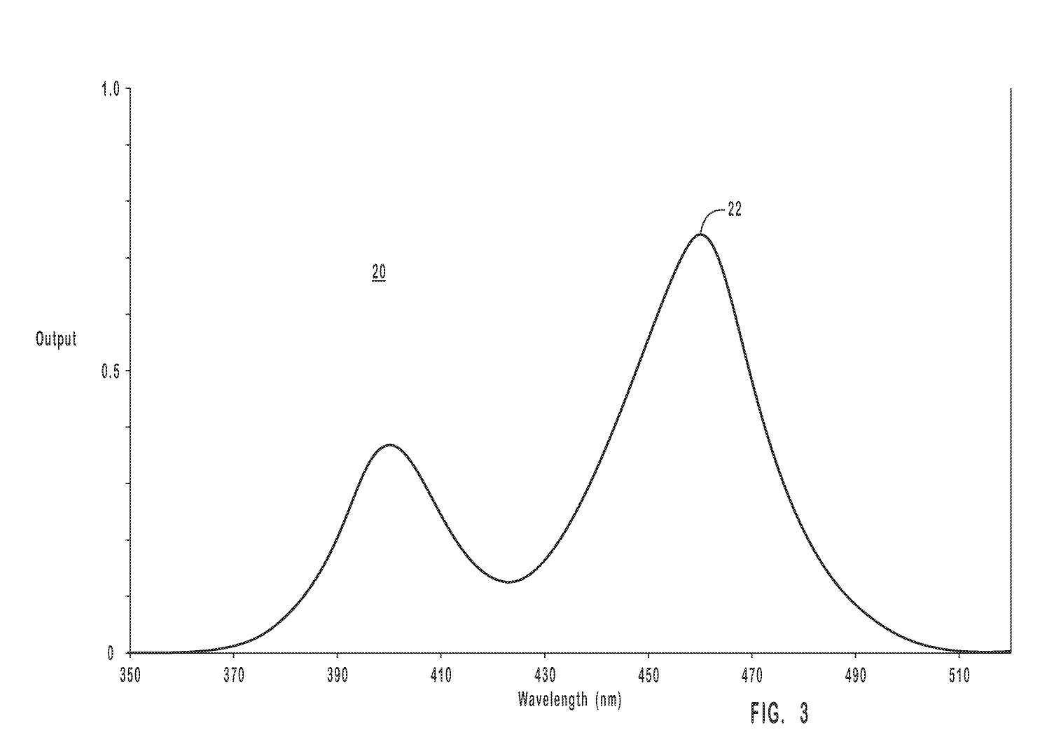

FIG. 3 illustrates a graph 20, illustrating the spectral irradiance of an exemplary dental curing light including a single LED configured to emit a first spectrum of light having a first relatively short peak wavelength (e.g., 380 nm) and a fluorescing lens. Spectral irradiance 22 includes a first peak at about 380 nm and a second peak at about 460 nm. In the illustrated graph, a portion of the 380 nm light produced by the LED is converted by a fluorescing lens into a second spectrum of light having a longer peak of about 460 nm. Such a dental curing light is useful for curing both camphorquinone initiated products and UV light initiated adhesives as the spectral irradiance 22 includes both a UV component (i.e., a 380 nm peak) and a blue component (i.e., a 460 nm peak). Although the graph illustrates only a portion of the shorter wavelength light (i.e., 380 nm) being converted to a longer wavelength (i.e., 460 nm), it is to be understood that the fluorescing lens may be configured to convert all or substantially all of the shorter wavelength light into a longer wavelength, as desired.

Furthermore, such a dental curing light including a single relatively short wavelength LED and a fluorescing lens provides a more uniform intensity of the various peak wavelengths (e.g., 380 nm and 460 nm) across substantially the complete footprint of light emitted because there are not multiple point sources of light, as opposed to a dental curing light including multiple LEDs configured to emit at different wavelengths (e.g., a 380 nm LED and a 460 nm LED). In other words, “hot” and “cold” spots within the footprint are minimized or eliminated.

FIG. 4 illustrates an alternative dental curing light 200 including an elongate wand body 202 having a proximal end 204 and a distal end 206. The illustrated embodiment of dental curing light 200 includes two LEDs 208 configured to emit a first spectrum of light having a relatively short first peak wavelength, and a fluorescing lens 210. Fluorescing lens 210 converts at least a portion of the first spectrum of light emitted by LEDs 208 into longer wavelength light having a spectrum selected so as to match the curing spectrum of a desired light curable composition. LEDs 208 may be configured to emit light at the same wavelength or alternatively they may be configured to emit two different wavelengths, as desired.

The one or more LEDs may be configured to emit a first spectrum of light having a relatively short first peak wavelength having any peak wavelength, as desired. In one example, the LEDs are configured to emit a first spectrum of light having a first peak wavelength between about 350 nm and about 410 nm. It is to be understood that the LED or LEDs may emit light having a first peak wavelength less than 350 nm, if desired.

According to an alternative embodiment, the one or more LEDs are configured to emit a first spectrum of light having a relatively short first peak wavelength between about 350 nm and about 490 nm. It is to be understood that the LED or LEDs may emit light having a first peak wavelength less than 350 nm, if desired.

fluorescing lens converts at least a portion of the first spectrum of light into longer wavelength light of a second spectrum selected so as to match the curing spectrum of a desired light-curable dental composition. In one embodiment, the fluorescing lens may convert at least a portion of the shorter wavelength light into longer wavelength light having a second peak wavelength between about 350 nm and about 490 nm. In one such embodiment, the fluorescing lens converts at least a portion of the shorter wavelength light into a longer wavelength light having a second peak wavelength between about 350 nm and about 410 nm. Such an embodiment is useful for curing UV light activated adhesives. In another example, the fluorescing lens converts at least a portion of the shorter wavelength light into longer wavelength light having a second peak wavelength between about 430 nm and about 490 nm, preferably between about 440 nm and about 480 nm. Such an embodiment is useful for curing at least camphorquinone initiated products.

In order to convert shorter wavelength light into longer wavelength light, the fluorescing lens is preferably impregnated with, coated or otherwise made using one or more fluorescing dyes, pigments or other compounds that are able to absorb shorter wavelength light and then emit longer wavelength light. An example of a class of fluorescing compounds is fluorizine. A variety of fluorescing compounds sold under the trade name Edgeglo® are available from PolyOne Corporation.

Two or more different fluorescing compounds can be used, e.g., mixed together or layered to get a blended effect or within different sections of the lens to yield outputs at different wavelengths. In one example, at least a portion of the lens may include a light absorbing dye or pigment that filters all or some of the light emitted by the LED that would otherwise be transmitted by the lens (e.g., to filter out short wavelength light not converted by the fluorescing compound into longer wavelength light).

III. Exemplary Kits

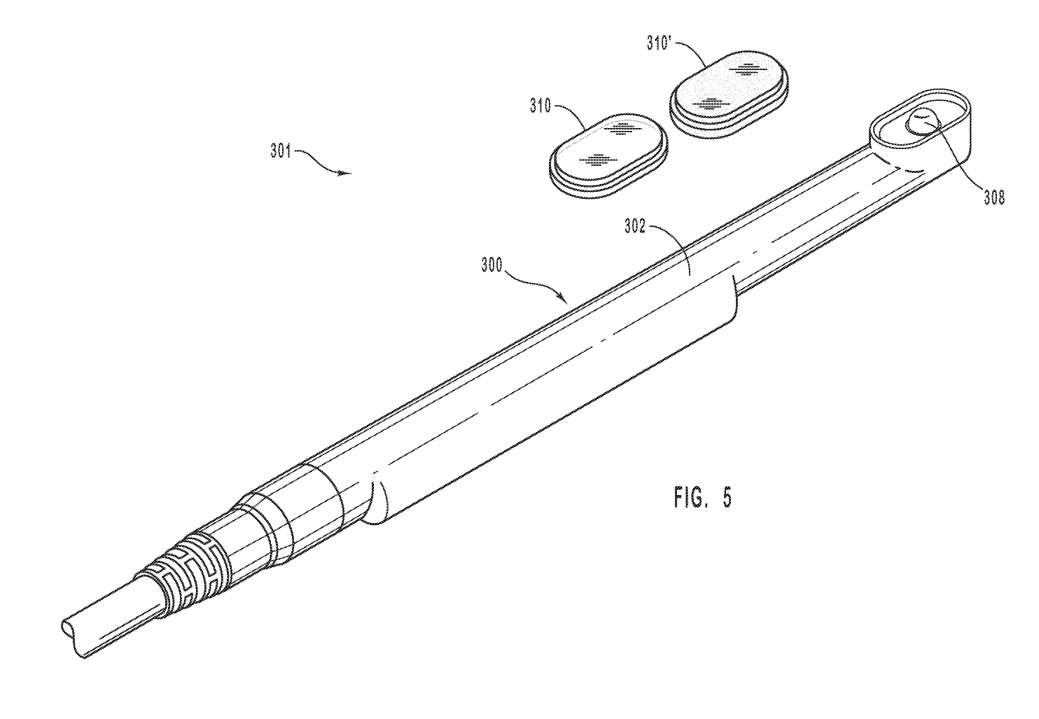

A kit 301 according to the present invention may include a dental curing light 300 and a plurality of different fluorescing lenses 310, 310′, as illustrated in FIG. 5. The dental curing light 300 includes a body 302 and one or more LEDs 308 disposed on the body 302. The LED or LEDs 308 are configured to emit a first spectrum of light having a relatively short first peak wavelength. The kit 301 includes a first fluorescing lens 310 that converts at least a portion of the first spectrum of light into a second spectrum of light having a second peak wavelength that is longer than the first peak wavelength. The second spectrum of light is selected so as to match the curing spectrum of a first light-curable dental composition. The kit 301 also includes at least a second fluorescing lens 310′ that converts at least a portion of the first spectrum of light into a third spectrum of light having a third peak wavelength that is longer than the first peak wavelength. The third spectrum of light is selected so as to match the curing spectrum of a second light-curable dental composition different from the first light-curable dental composition. Such a kit 301 may allow a user to interchange the first and second fluorescing lenses 310 and 310′ for use with the dental curing light 300, as desired.

In one example, at least one of the fluorescing lenses converts at least a portion of the first spectrum of shorter wavelength light into longer wavelength light having a peak wavelength between about 350 nm and about 410 nm. Such a lens is useful for curing light-curable dental compositions that include a UV light activated initiator. In another example, one of the fluorescing lenses may convert at least a portion of the first spectrum of shorter wavelength light into longer wavelength light having a peak wavelength between about 430 nm and about 490 nm. Such a lens may be useful for curing a light-curable dental composition including camphorquinone as an initiator. If the lens only converts a portion of the light emitted by the LED, the dental curing light and lens may also be useful for curing light-curable dental compositions that include a UV light activated initiator, depending on the wavelength of light emitted by the LED or LEDs.

The fluorescing lenses are preferably interchangeable and selectively attachable and detachable from the body of the dental curing light, so as to allow a user to interchange the lenses as desired. Such a kit may further include one or more light-curable dental compositions. For example, a light curable dental composition may be provided which can be cured by use of one the provided fluorescing lenses.

IV. Exemplary Method of Use

According to one exemplary method, the dental curing light may be used to cure a desired light-curable dental composition. A dental curing light as described above is provided, and a fluorescing lens that converts at least a portion of the relatively short first spectrum of light emitted by the dental curing light LED is then selected. The fluorescing lens is specifically selected for its ability to convert the relatively short wavelength light into longer wavelength light having a spectrum that matches the curing spectrum of a desired light-curable dental composition. The selection of the appropriate fluorescing lens is made according to the curing spectrum requirements of a light-curable dental composition to be cured. The selected fluorescing lens is then attached to the dental curing light, and the dental curing light is used with the fluorescing lens to cure a desired light-curable composition.

It will be appreciated that the present claimed invention may be embodied in other specific forms without departing from its spirit or essential characteristics. The described embodiments are to be considered in all respects only as illustrative, not restrictive. The scope of the invention is, therefore, indicated by the appended claims rather than by the foregoing description. All changes that come within the meaning and range of equivalency of the claims are to be embraced within their scope.

What is claimed is:

1. A dental curing light comprising:
a body;
one or more LEDs disposed on the body, the LEDs being configured to emit a first spectrum of light having a first wavelength suitable for curing a dental composition; and
a fluorescing lens that converts at least a portion of the first spectrum of light into a second spectrum of light having a second wavelength longer than the first wavelength.

2. A dental curing light as recited in claim 1, wherein the body comprises an elongate wand having a proximal end and a distal end.

3. A dental curing light as recited in claim 2, wherein the one or more LEDs are disposed at or near the distal end of the elongate wand.

4. A dental curing light as recited in claim 1, wherein the one or more LEDs are configured to emit a first spectrum of light having a peak wavelength within a UV range.

5. A dental curing light as recited in claim 1, wherein the one or more LEDs are configured to emit a first spectrum of light having a peak wavelength between about 350 nm and about 490 nm.

6. A dental curing light as recited in claim 1, wherein the second spectrum is of light is selected to cure light curable dental compositions comprising at least one of camphorquinone or light curable dental compositions that are cured by light having a peak wavelength of less than 490 nm but greater than 350 nm

7. A dental curing light as recited in claim 1, wherein the fluorescing lens converts at least a portion of the first spectrum of light into a second spectrum of light having a peak wavelength between about 350 nm and about 410 nm.

8. A dental curing light as recited in claim 1, wherein the fluorescing lens converts at least a portion of the first spectrum of light into a second spectrum of light having a peak wavelength between about 430 nm and about 490 nm.

9. A dental curing light as recited in claim 1, wherein the fluorescing lens is a first selectively attachable and detachable lens for selective attachment or removal from the body of the dental curing light.

10. A dental curing light as recited in claim 1, wherein the fluorescing lens converts only a portion of the first spectrum of light into a second spectrum of light.

11. A dental curing light as recited in claim 1, wherein the fluorescing lens converts substantially all of the first spectrum of light into a second spectrum of light.

12. A dental curing light as recited in claim 9, further comprising a second fluorescing lens that is selectively attachable and detachable and which may be used to selectively replace the first fluorescing lens, and wherein the second fluorescing lens converts at least a portion of the first spectrum of light into a third spectrum of light having a third peak wavelength that is longer than the first peak wavelength, but which is different from the second spectrum of light produced by the first fluorescing lens.

13. A dental curing light as recited in claim 12, wherein the first fluorescing lens converts at least a portion of the first spectrum of light into a second spectrum of light having a peak wavelength between about 350 nm and about 410 nm.

14. A dental curing light as recited in claim 12, wherein the second fluorescing lens converts at least a portion of the first spectrum of light into a third spectrum of light having a peak wavelength between about 430 nm and about 490 nm.

15. A dental curing light comprising:
a body in the form of an elongate wand having a proximal end and a distal
one or more LEDs disposed at or near the distal end of the elongate wand, the LEDs being configured to emit a first spectrum of light having a first peak wavelength suitable for curing a dental composition; and
a fluorescing lens attachable over the one or more LEDs, the fluorescing lens converting at least a portion of the first spectrum of light into a second spectrum of light having a second wavelength longer than the first wavelength.

16. A dental curing light as recited in claim 15, wherein the fluorescing lens converts only a portion of the first spectrum of light into a second spectrum of light having a peak wavelength between about 440 nm and about 480 nm.

17. A dental curing light as recited in claim 15, wherein the one or more LEDs comprise only a single LED such that the single LED and the fluorescing lens provide a substantially uniform intensity of the first and second peak wavelengths across a footprint of light emitted by the dental curing light.

18. A dental curing light as recited in claim 15, wherein the fluorescing lens converts only a portion of the first spectrum of light into a second spectrum of light having a second peak wavelength between about 430 nm and about 490 nm, and such that the dental curing light is selectably configured to cure both light curable dental compositions comprising camphorquinone as well as light curable dental compositions comprising a UV light activated photo-initiator.

19. A method comprising:
providing a dental curing light comprising:
a body; and
one or more LEDs disposed on the body, the LEDs being configured to emit a first spectrum of light having a first peak wavelength suitable for curing a dental composition;
selecting a first fluorescing lens that converts at least a portion of the first spectrum of light into a second spectrum of light having a second peak wavelength that is longer than the first peak wavelength;
attaching the selected fluorescing lens to the dental curing light; and
using the dental curing light and attached fluorescing lens to produce a second peak wavelength that is longer than the first peak wavelength for use in performing a dental procedure.

20. A method as recited in claim 19, wherein the dental curing light and attached fluorescing lens are used to cure a camphorquinone initiated light curable composition.

21. A method as recited in claim 19, wherein the dental curing light and attached fluorescing lens are used to cure a UV initiated light curable composition.

22. A method as recited in claim 19, wherein a second fluorescing lens is selectively attached to replace the first fluorescing lens, and wherein the second fluorescing lens converts at least a portion of the first spectrum of light into a third spectrum of light having a third peak wavelength that is longer than the first peak wavelength, but which is different from the second spectrum of light produced by the first fluorescing lens.
